(19) United States
(12) Patent Application Publication
(10) Pub. No.: US 2021/0379001 A1
(43) Pub. Date: Dec. 9, 2021

(54) SUPPLEMENT INCLUDING GAMMA-AMINOBUTYRIC ACID FOR RESTORING GROWTH HORMONE LEVELS

(71) Applicant: 1198966 B.C. LTD., Peachland (CA)

(72) Inventor: Philip Anthony WHITE, Kelowna (CA)

(21) Appl. No.: 17/339,468

(22) Filed: Jun. 4, 2021

Related U.S. Application Data
(60) Provisional application No. 63/035,140, filed on Jun. 5, 2020.

(30) Foreign Application Priority Data
Jun. 5, 2020 (CA) 3081988

(51) Int. Cl. A61K 31/198; A61K 31/197

(52) U.S. Cl. CPC A61K 31/198; A61K 31/197

(57) ABSTRACT

A nutritional supplement for ingestion by humans for restoring growth hormone levels consisting of branched chain amino acids chosen from the group leucine, isoleucine, and valine; free form amino acids chosen from the group lysine, glutamine, ornithine, arginine, and glycine; and free form amino acid gamma-aminobutyric acid.

### CROSS-REFERENCE TO RELATED APPLICATIONS

The present application claims priority to U.S. provisional patent application No. 63/035,140 and Canadian patent application no. 3,081,988, both of which were filed on Jun. 5, 2020, the entirety of which are incorporated herein by reference.

### FIELD

This disclosure relates to the field of nutritional supplements, including supplements which elevate release of human growth hormone.

### BACKGROUND

It is the Applicant's understanding that what follows in the Background section of the present disclosure is accepted by at least some experts in the field of nutritional supplements.

It is accepted that higher levels of circulating growth hormone (hGH) in humans, along with cellular resilience factors (the ability to recover from stresses), determines “biological age.” Biological age is essentially the age of your body's cells, independent of chronological age. Decreasing biological age increases life span. hGH and cellular resilience factors can be influenced by lifestyle choices, diet, and nutritional supplementation. Lifestyle and diet are controlled by individual choice. Thus, the opportunity to impact human health and longevity exists by uniquely formulating a nutritional supplement, designed to maximize levels of circulating hGH and speed cellular recovery from the effects of stress; disease, lack of sleep, fear/anxiety, and physical exertion.

Until recently human growth hormone (hereinafter alternatively referred to as hGH) was available only in expensive injectable forms, and the benefits from the restoration of hGH levels was available only to those with the ability to pay. Most recently natural substances which can trigger the release of human growth hormone from an individual's own anterior pituitary gland have become available. These are generically referred to as secretagogues. Secretagogues have the ability to restore hGH levels, potentially to the levels found in youth. See for reference the book entitled “Grow Young With hGH” by Dr. Ronald Klatz, President of the American Academy of Anti-Aging, published in 1997 by Harper Collins.

A study, published in 1981 by Drs. Isidori, A., Lo Monica and Cappa (Isidori, A., et al. Current Medical Research and Opinion 7 # 7 (1981): 475-481), demonstrated that a specific combination of amino acids, when ingested orally, would cause an increase in growth hormone levels in humans. It offered a more practical and physiological approach to the previously known fact that intravenous administration of amino acids strongly stimulates the secretion of human growth hormone by the anterior pituitary gland.

Franco Salomon et al [The New England Journal of Medicine Vol. 321 (26) p. 1797-1803) 1989] carried out a six month randomized, double blind, placebo controlled trial of recombinant human growth hormone on 24 patients suffering from growth hormone deficiency. They noticed an increase in Insulin-like Growth Factor 1 (“IGF-1”), lean body mass and reduction in fat. Metabolic rate was increased and plasma cholesterol lowered.

JO. L. Jorgensen et al (Lancet-Jun. 3^{rd}, 1989 p. 1221-1225) carried out a 4 month double blind, placebo controlled, crossover study of growth hormone in 22 deficient adults. Muscle thickness increased, fat was reduced. Renal plasma flow and glomerular filtration rates were raised from subnormal levels to levels comparable for their age. IGF-1 levels were also normalized.

In 1990 Dr. Daniel Rudman published a research paper (Rudman, D., et al. “Effects of Human growth Hormone in men over 60 years Old”, New England Journal of Medicine, 323 (1990): 1-6) which showed that twelve healthy men aged between 61 and 81, following six months of human growth hormone therapy, had age reversal effects on lean body mass and adipose tissue mass equivalent in magnitude to changes attributable to ten to twenty years of aging. In addition, Dr. Rudman et al noted that alterations in body composition, caused by growth hormone deficiency as we age, can be reversed by replacement doses of hormone in other experiments in rodents, children and adults 20 to 50 years old. Dr. Rudman also noted that “these findings suggest that the atrophy of the lean body mass and its (the body's) component organs and the enlargement of the mass of adipose tissue that are characteristic of the elderly results at least in part from diminished secretion of growth hormone. If so, the age-related changes in body composition should be correctable in part by the administration of human growth hormone, now readily available as a bio-synthetic product”.

The primary purpose of hGH is that of stimulating growth, cell repair and regeneration. Once the growth period is over, its primary function becomes that of cell regeneration and repair, helping to regenerate skin, bones, heart, lungs, liver and kidneys to their former youthful cell levels. Elevating hGH levels appears to benefit the immune system. It has also been reported the risk factors for heart attack and stroke may be potentially diminished. Some patients with emphysema have reported that they are less short of breath. Dr. Rudman's study also demonstrated that bone density in the lumbar spine can improve. It has also been shown that wrinkled skin diminishes. Others have reported improvement in presbyopia (the difficulty in focussing due to hardening of the lenses as we age) with some reporting restoration of hair colour and growth. hGH appears to selectively reduce the fat around the abdomen, hips, waist and thighs while at the same time increasing muscle mass. In Dr. Rudman's study, after six months of usage, without exercise, the subjects had an average 8% increase in lean muscle mass and a 14.4% loss of fat.

Dr. Cass Terry and Dr. E. Chein reported on the effects of elevating hGH levels by injection. They showed high frequency-low dose injections of hGH elevated IGF-1 levels. Analyzing the clinical effects of elevating hGH in 202 patients (age 39-74) they showed 75% of the individuals described an increase and improvement in sexual potency as well as frequency of sexual relations, while 62% described improvement in duration of penile erection. Energy levels were improved in 84% of individuals and muscle strength improved in 88%. A 71% increase in healing capacity was noted.

It appears that hGH not only has the ability to restore sexual potency and sexuality in older men, but acts as a mood elevator, restoring a youthful sense of wellness as well as improving sleep. With its potential for affecting cell regeneration and repair, healing of injuries should improve.

The hormone hGH, alternatively referred to herein by its medical name Somatotropin, is produced in the anterior pituitary gland situated just below the hypothalamus, which is itself situated just below the cerebral cortex of the brain. hGH is one of several hormones secreted by the anterior pituitary gland and, as noted above, influences the growth, regeneration and repair of cells, bones, muscles and organs throughout the body throughout life. Production peaks at adolescence when accelerated growth is occurring, and if growing children are deficient in human growth hormone, they remain as pituitary dwarfs. If they have too much they exhibit gigantism (acromegaly).

As is the case with many of our other hormones or their pre-cursors, such as testosterone, oestrogen, progesterone, DHEA and melatonin, hGH levels decline with age. Therapeutically, many of these hormones can be replaced to offset some of the effects of aging such as menopausal symptoms in women or erectile dysfunction in men.

The human body, like every other living entity, works on a daily, or circadian as well as monthly and annual rhythms. Daily growth hormone secretion diminishes with age with roughly half the levels at age forty that we had when we were twenty, and about one-third of those youthful levels at age sixty. In some sixty-year olds the levels are as low as 25% of the hGH levels in a twenty-year old. Symptoms of aging include loss of muscle, increase of fat, decreased physical mobility, decreased energy levels and as a result, diminished socialization, diminished healing ability and an increased risk of cardiovascular disease and decreased life expectancy.

Low hGH levels are associated with the aging process and early onset of disease. For example, Rosen and Bengtsson noted an increased death rate from cardiovascular disease in hGH deficient patients (Rosen, T., Bengtsson, B. A., Lancet 336 (1990): 285-2880). Furthermore, the mechanism of hGH release has been elucidated and is considered to be under the control of Growth Hormone Releasing Hormone (GHRH) and Somatostatin. Somatostatin prevents further release of hGH from the pituitary gland. It has been postulated that one of the key factors in aging is an imbalance in the levels of GHRH and Somatostatin in the combined GHRH/Somatostatin secretion, with an increased action or effectiveness of Somatostatin over GHRH. This leads to an effective reduction in release of the stored hGH from the anterior pituitary gland. lsidori et al have shown the selected amino acids arginine and lysine increase the release of the body's own stored, natural hGH, when taken orally. Matteini showed even low doses of arginine in the region of 200 mg can elevate hGH release. (Matteini, M., et al. Bollettino-Societa Italiana Biologia Sperimentale. 56(21): 2254-60, Nov. 15, 1980). It has been suggested one of the mechanisms of action is the inhibitory effect of arginine, and possibly other amino acids, on the secretion and action of Somastostatin (Alba-Roth, J., Muller, O. A., Schopohl, J. et al. Journal of Clinical Endocrinology and Metabolism 67, #6 (1998): 1186-1189).

hGH, once released by the pituitary gland, travels in the circulation and is taken up principally by the liver where it stimulates the production of IGF-1. IGF-1 is then released into the circulation where it attaches to cells in the body and like insulin, triggers the cell to produce certain responses which, with IGF-1, are those of growth, regeneration and repair. Levels of IGF-1 are monitored by the hypothalamus situated just above the pituitary gland. When maximal hGH levels are attained for any individual, these levels trigger the release of Somatostatin. This feedback loop prevents excessive levels of hGH in the body. This feedback loop is extremely efficient at monitoring and maintaining the hGH (and therefore IGF-1) at the optimal level for the individual.

In the prior art, U.S. Pat. No. 6,346,264 to Philip White discloses a nutritional supplement comprising an amino acid stack secretagogue which, taken orally, stimulates the pituitary gland to produce hGH/Somatropin, so as to elevate hGH/Somatropin release.

The amino acid gamma-aminobutyric acid (“GABA”) is known in the prior art, and was recently approved for use in human nutritional supplements. GABA is a non-protein amino acid inhibitory neurotransmitter, not generally found in foods. Oral administration of the amino acid/inhibitory neurotransmitter gamma aminobutyric acid (GABA) reportedly elevates serum growth hormone (hGH) concentrations. In addition to stimulating hGH production, GABA is known to calm anxieties, enhance sleep and muscle mass retention, along with other anti-aging benefits in humans. However, when GABA alone is orally ingested as a dietary supplement, it confers limited benefits to humans.

The interaction between GABA and other amino acids has been studied. For example, the possibility that circulating glutamine affects GABA levels in rat striatal tissue and GABA concentrations in in striatal extracellular fluid, in a paper published in 2007 (Wang et al, FASEB Journal, April 2007; 21(4): 1227-32.) L-Glutamine administration significantly increased ECF GABA concentrations by 30%, and enhanced the response evoked by NMDA alone (70%) to 120% over baseline (all P<0.05). Striatal GABA levels increased significantly 2,5 h after oral L-glutamine (e.g., from 1.76+/−0.04 micromol/g in vehicle-treated rats to 2.00+/−0.15 micromol/g in those receiving 2.0 g/kg of glutamine), Striatal glutamine levels also increased significantly, but not those of glutamate. These data suggest that GABA synthesis in, and release from, rat striatum may be regulated in part by circulating glutamine. Hence, glutamine administration may provide a useful adjunct for treating disorders (e.g., anxiety, seizures) when enhanced GABAergic transmission is desired. Moreover, the elevation in plasma and brain glutamine associated with hepatic failure may, by increasing brain GABA release, produce some of the manifestations of hepatic encephalopathy.

In an article published in 2019, the effects of the combination of whey protein and GABA supplementation in men was studied in a group of men to determine whether the combination of whey protein and GABA supplementation increased muscle mass in combination with exercise (Sakashita et al, J Clin Med Res. 2019 Jun; 11(6): 428-434). In the group that orally ingested whey protein and GABA, resting plasma growth hormone concentrations were significantly elevated at four and eight weeks, compared to baseline. However, resting plasma growth hormone concentrations, in the group that orally ingested whey protein alone, were only significantly elevated at eight weeks. After 12 weeks, the group that took whey protein and GABA exhibited significantly greater increase in whole body fat-free mass than the group that took whey protein alone. The paper's authors concluded that the GABA and whey protein combination was more effective for increasing whole body fat-free mass, and that daily GABA supplementation may help enhance exercise-induced muscle hypertrophy. The Applicant has noted that different brands of whey protein contain varying mass ranges of various different amino acids in one serving of the whey protein.

### SUMMARY

Oral administration of the amino acid/inhibitory neurotransmitter gamma aminobutyric acid (GABA) reportedly elevates serum growth hormone (hGH) concentrations. Likewise, a nutritional supplement, ingested by humans for restoring hGH levels, consists of branched chain amino acids chosen from the group leucine, isoleucine, and valine, and free form amino acids chosen from the group lysine, glutamine, ornithine, arginine, and glycine. This supplement is an amino acid stack secretagogue that stimulates the secretion of hGH by the anterior pituitary gland.

In one aspect of the present disclosure, the applicant has found that combining GABA and the aforementioned amino acid stack secretagogue to form a single, oral nutritional supplement demonstrates increased effectiveness of the benefits of using GABA alone or the amino acid stack secretagogue alone. Notwithstanding that the Applicant does not wish to be held to any particular theory, the Applicant postulates that the combination of GABA with the aforementioned amino acid stack secretagogue may catalyze levels of circulating hGH and activates high levels of cellular resilience factors.

The benefits of an amino acid stack secretagogue, combined with GABA in an ingested nutritional supplement, taken orally, optimally stimulates the pituitary gland to elevate hGH/Somatotropin release and increase IGF-1 levels because of their complimentary structures and different mechanisms of action. The secretagogues' natural feedback loop mimics the body's youthful hGH secretion patterns, restoring the body's hGH and IGF-1 levels without interference. The types of amino acids, mass ranges and specific formulations disclosed herein produce a synergistic balance for peak effectiveness; and when combined, the secretagogue catalyzes the GABA, thereby maintaining consistent glucose levels which in turn boosts the body's ability to elevate hGH levels and increase hGH production.

In combination with the other physiological effects of orally ingested GABA, the benefits to increasing hGH are many, including: inhibiting insulin depression, reducing hyperglycemia, and increasing insulin effectiveness, enhancing fat conversion, assisting in lowering cholesterol, normalizing lipid balance, revitalizing skin, hair and nails, improving quality of sleep, elevating mood, and increasing energy.

In one aspect, the present disclosure relates to a nutritional supplement. It is an amino acid stack secretagogue which includes GABA and a selection of other amino acids, which, taken orally, stimulates the pituitary gland to produce hGH/Somatotropin. One object of the present disclosure is to elevate hGH/Somatotropin release. This has the further result of increasing IGF-1 levels. Further objects of the present disclosure may also result in the following physiological effects induced by taking the nutritional supplement disclosed herein, including: inhibiting insulin depression; inhibiting hyperglycemia and increasing insulin effectiveness; enhancing fat conversion; assisting in lowering cholesterol; and normalizing lipid balance. Particular benefits observed from taking the nutritional supplement, incorporating GABA and a selection of other amino acids, may include, but are not limited to, improved sleep quality, increased weight loss, increased energy throughout the day, improved nail growth and structure, and/or improved mood.

In summary, the improved nutritional supplement for restoring growth hormone levels of the present disclosure comprises in combination, or, alternatively, consists essentially of in combination the following free amino acids (alternatively in the “L” form such as L-Glutamine): lysine, ornithine, glutamine, glycine, leucine, iso-leucine, valine and gamma-aminobutyric acid; and may also include arginine.

In one aspect, the present disclosure is a nutritional supplement for ingestion by humans for restoring growth hormone levels including or alternatively consisting essentially of:
(a) branched chain amino acids chosen from the group leucine, isoleucine, and valine;
(b) free form amino acids chosen from the group lysine, glutamine, ornithine, arginine, and glycine; and
(c) gamma-aminobutyric acid.

In a further aspect of the present disclosure, the nutritional supplement for ingestion by humans for restoring growth hormone levels includes or alternatively may consist essentially of all of the amino acids leucine, isoleucine, valine, lysine, glutamine, ornithine, glycine and gamma-aminobutyric acid; and may also include arginine.

In a daily dosage the nutritional supplement may include or alternatively consist essentially of:
(a) 1302 mg of L-Lysine HCl;
(b) 1302 mg of L-Arginine HCl;
(c) 798 mg of L-Ornithine HCl;
(d) 500 mg of Glycine;
(e) 250 mg of L-Glutamine;
(f) 400 mg of L-Leucine;
(g) 400 mg of L-Isoleucine;
(h) 400 mg of L-Valine; and
(i) 100 mg of GABA.

In alternative formulations, a daily dosage of the nutritional supplement will include, or consist essentially of:
(a) Lysine in the mass range 500 mg and 1500 mg;
(b) Arginine in the mass range 500 mg and 1500 mg;
(c) Ornithine in the mass range 500 mg and 1500 mg;
(d) Glutamine in the mass range 200 mg and 1000 mg;
(e) Glycine in the mass range 400 mg and 1000 mg;
(f) Leucine in the mass range 400 mg and 1000 mg;
(g) Iso-leucine in the mass range 400 mg and 1000 mg;
(h) Valine in the mass range 400 mg and 1000 mg; and
(i) GABA in the mass range 100 mg and 200 mg.

### DETAILED DESCRIPTION

As noted above, elevating hGH levels elevates IGF-1 levels. This can be achieved by secretagogue hGH releasers without the need for injection therapy or oral-hGH forms, or gene manipulation. Secretagogues maintain the body's own natural feedback loop, thus not only releasing hGH naturally but safely. Natural secretagogues may have the ability to more closely mimic the body's youthful hGH secretion patterns than any other hGH therapies currently available. Use of a secretagogue such as the supplement of the present disclosure has the potential for restoring the body's hGH and IGF-1 levels without interfering with the body's own feedback loop. The supplement of the present disclosure contains amino acids, formulated as a dietary supplement.

Some amino acids can be made in the body from basic building blocks, but others (nine out of the twenty needed for protein building) are called essential amino acids as the body is unable to manufacture them and they have to be supplied in the food that we eat. Arginine is an amino acid important in creating synthesis. Claims for Arginine include an increase in fat-burning and muscle-building, as well as strengthening the thymus gland by increasing its weight and activity and thereby boosting immunity. There are also claims that Arginine alone will promote healing of burns and wounds as well as enhance male fertility.

Arginine along with Lysine have been demonstrated to cause hGH release when combined in specific proportions. Essential amino acids contained in the supplement of the present disclosure are Isoleucine, Leucine, Lysine and Valine. Lysine boosts the effectiveness of Arginine and is also said to affect growth as well as having immune-boosting properties of its own. Ornithine can be synthesized in the body and is now also known to help stimulate hGH release. Glutamine can also be synthesized in the body but may not always be made by the body in sufficient quantities in times of stress. Without sufficiently available levels, the gastrointestinal tract does not function as well, and nutrients are less well absorbed. GABA, which in recent years has been approved for use in human nutritional supplements, is also known to act as an effective hGH releaser and confers a relaxing, calming effect, somewhat similar to the effects of the amino acid tryptophan. Both GABA and tryptophan are neurotransmitter precursors, and may be effective in treating anxiety and stress. Other amino acids in the supplement of the present disclosure contribute to the effects of the supplement and the synergy of the amino acids listed above. This type of combination of amino acids is known as an amino acid stack.

The supplement of the present disclosure works as a dietary supplement by assisting the body's own ability to secrete hGH naturally in a manner which is safe and effective as well as being affordable. In one aspect, when starting the supplement of the present disclosure it should be taken for a minimum of three months, preferably along with a dietary and exercise regime in order to ensure maximal benefits. Continued usage is suggested for maximum benefit and it is also recommended that it be taken on a six month cycle followed by one month off, or six days per week with one day off, based on our current state of knowledge. The supplement of the present disclosure may be formulated in a capsule form for ease of ingestion. It should be taken on an empty stomach, either before bed or first thing in the morning, for best results. This ensures that it is rapidly absorbed into the bloodstream.

What is not taught nor suggested in the prior art, and which the supplement of the present disclosure provides is the use, in the below disclosed combination and proportions, of amino acids including GABA, lysine, glutamine and ornithine, alternatively to also include arginine, to inhibit insulin depression, often seen when hGH levels are elevated. The supplement of the present disclosure in the specified combination and having the specified quantity of glutamine inhibits hyperglycemia, while ensuring a natural anabolic muscle building effect by increasing insulin effectiveness. In the supplement of the present disclosure ornithine is combined with glutamine to enhance fat conversion and assist in lowering cholesterol, while normalizing lipid balance. In the supplement of the present disclosure, in the specified combination and quantities, amino acids are stacked amino acid secretagogues resulting in balancing glucose, insulin and blood lipids. The supplement of the present disclosure includes the branched-chain amino acids leucine, isoleucine and valine so as to provide essential nutrients for muscle regeneration and repair. This is of particular importance when a secretagogue is used before exercise.

Although the oral administration of GABA, ingested as a dietary supplement, is known to have benefits for human health, including the stimulation of hGH production, reducing anxiety, enhancing sleep and increased muscle mass retention, amongst other anti-aging health benefits, the use of GABA alone in a dietary supplement has not been shown to confer significant benefits to human health. GABA, as a non-protein amino acid inhibitory neurotransmitter, is not generally found in foods. However, the applicant has discovered that the known benefits of GABA, when taken in an oral dietary supplement that combines GABA with an amino acid stack secretagogue, the dietary supplement optimally stimulates the pituitary gland to elevate hGH/Somatropin release and increases IFG-1 levels because of the complimentary structures and different mechanisms of action.

As a result of combining GABA with the amino acid stack secretagogue disclosed herein, the applicant has found that the beneficial effects of GABA on the human body are enhanced, as compared to a supplement that contains GABA alone. The dietary supplement that combines GABA with the amino acid stack secretagogue, as described in the present disclosure with respect to the specific combination of amino acids, their mass ranges and specific formulations, provides for a synergistic balance for enhanced effectiveness of the supplement, and the amino acid stack secretagogue beneficially catalyzes GABA while maintaining consistent glucose levels in the body, which in turn boosts the body's ability to elevate hGH levels and increase hGH production. As a result, the secretagogues' natural feedback loop mimics the body's youthful hGH secretion patterns, restoring the body's hGH and IGF-1 levels without interference.

In combination with GABA, the benefits of increasing hGH production are many, and may include inhibiting insulin depression, reducing hyperglycemia and increasing insulin effectiveness, enhancing fat conversion, assisting in lowering cholesterol, normalizing lipid balance, revitalizing skin, hair and nails, improving quality of sleep, elevating mood and increasing energy, amongst other benefits.

The combination of types of amino acids, mass ranges and specific formulations have been selected to be synergistically balanced and of adequate quantity to achieve the desired physiological effect, namely, growth hormone release. Improper combinations of the amino acids or insufficient quantities of amino acid salts will be ineffective. The component amino acids are synergistic in the sense that several of them when combined together, including GABA, arginine, lysine, glutamine, ornithine and glycine, synergistically stimulate the release of human growth hormone. The combination was also chosen to inhibit chemical combination or reaction between the amino acids. Such will not occur because of the crystalline free-form amino acid salts that have been chosen.

A further novel aspect of the supplement of the present disclosure is the addition of the sulphur-containing branch-chain amino acids leucine, iso-leucine and valine, all of which themselves have a synergistic effect in combination with the human growth hormone that is released in helping to build and improve muscles mass and strength.

With regard to the individual amino acids in particular, ornithine is synergistic with arginine in elevating hGH levels as is lysine synergistic with arginine in elevating hGH levels. Glutamine will elevate hGH levels independently and there is some evidence of a synergistic effect with the three others mentioned above with glycine also producing independent induction of hGH secretion and synergy with the others. The three sulphur-containing branch-chain amino acids, leucine, iso-leucine and valine all have been shown to promote muscle tissue repair and growth particularly in conjunction with human growth hormone as well as increased muscle energy and improvement in skin and wound healing, This in turn would of course help with exercise as vigorous exercise induces a mild level of muscle damage resulting in the aching effect that people notice when they have not been exercising for a while and start to exercise.

GABA is an inhibitory neurotransmitter in the brain. Too much excitation in the brain can lead to restlessness, irritability and insomnia. Advantageously, GABA is able to induce relaxation, analgesia and improve sleep cycles, leading to more restful sleep, which in turn assists with boosting the body's ability to elevate hGH levels because increasing the length and quality of sleep leads to increased hGH production.

Altogether the product is unique in its combination of amino acids in a free-form crystalline state, avoiding an interaction or chain reaction between the amino acids which could result in deterioration and breakdown in the integrity of the product. As well this form avoids the possibility of interaction with the vegetarian gel cap in which the product may be packaged.

In one preferred embodiment, the supplement of the present disclosure formulation for a one day dose, in 6-8 size 00 capsules, includes the following amino acids in the specified approximate mass ranges:
LYSINE 500 mg-1500 mg
ORNITHINE 500 mg-1500 mg
GLUTAMINE 200 mg-1000 mg
GLYCINE 400 mg-1500 mg
LEUCINE 400 mg-1000 mg
ISO-LEUCINE 400 mg-1000 mg
VALINE 400 mg-1000 mg
GABA 50 mg-300 mg

Another preferred embodiment may further include:
ARGININE 500 mg-1500 mg

Another preferred embodiment may include the amino acids lysine, ornithine, glutamine, glycine, leucine, iso-leucine and valine, in the approximate mass ranges specified above, with or without Arginine in the specified approximate mass range specified above, and GABA in the approximate mass range of 100 mg-200 mg.

In particular, in one preferred embodiment, the nutritional supplement of the present disclosure may have the following specific mass composition:
1302 mg of L-Lysine HCl;
1302 mg of L-Arginine HCl;
798 mg of L-Ornithine HCl;
500 mg of Glycine;
250 mg of L-Glutamine;
400 mg of L-Leucine;
400 mg of L-Isoleucine;
400 mg of L-Valine; and
100 mg of GABA.

As will be apparent to those skilled in the art in the light of the foregoing disclosure, many alterations and modifications are possible in the practice of this disclosure without departing from the spirit or scope thereof. Accordingly, the scope of the disclosure is to be construed in accordance with the substance defined by the following claims.

What is claimed is:

1. A nutritional supplement for ingestion by humans for restoring growth hormone levels consisting of unmodified rapidly absorbed free-form amino acids, in an amino acid stack which does not include non-amino acid nutritional supplementation, and which, when digested by a user, stimulates release of human growth hormone in the user, said amino acids consisting of:
branched chain amino acids chosen from the group:
(i) leucine,
(ii) isoleucine,
(iii) valine; and
(b) free form amino acids chosen from the group:
(i) lysine,
(ii) glutamine,
(iii) ornithine,
(iv) arginine,
(v) glycine; and
(c) free form amino acid gamma-aminobutyric acid.

2. A nutritional supplement for ingestion by humans for restoring growth hormone levels consisting of unmodified rapidly absorbed free-form crystalline amino acids, in an amino acid stack which does not include non-amino acid nutritional supplementation, and which, when digested by a user, stimulates release of human growth hormone in the user, wherein said amino acids consist of the amino acids leucine, isoleucine, valine, lysine, glutamine, ornithine, glycine and gamma-aminobutyric acid.

3. The nutritional supplement of claim 2, wherein the amino acids further consist of the amino acid arginine.

4. The nutritional supplement of claim 3, wherein said amino acids in the supplement are to be ingested by humans in a daily dosage, said amino acids in the daily dosage consist of:
(a) Lysine in the mass range 500 mg and 1500 mg;
(b) Arginine in the mass range 500 mg and 1500 mg;
(c) Ornithine in the mass range 500 mg and 1500 mg;
(d) Glutamine in the mass range 200 mg and 1000 mg;
(e) Glycine in the mass range 400 mg and 1000 mg;
(f) Leucine in the mass range 400 mg and 1000 mg;
(g) Iso-leucine in the mass range 400 mg and 1000 mg;
(h) Valine in the mass range 400 mg and 1000 mg; and
(i) Gamma-aminobutyric acid in the mass range 50 mg and 300 mg.

5. The nutritional supplement of claim 4, wherein the amino acid Gamma-aminobutyric acid is in the mass range 100 mg and 200 mg.

6. The nutritional supplement of claim 4, wherein said amino acids in the daily dosage consist of:
(a) 1302 mg of L-Lysine HCl;
(b) 1302 mg of L-Arginine HCl;
(c) 798 mg of L-Ornithine HCl;
(d) 500 mg of Glycine;
(e) 250 mg of L-Glutamine;
(f) 400 mg of L-Leucine;
(g) 400 mg of L-Isoleucine;
(h) 400 mg of L-Valine; and
(i) 100 mg of Gamma-aminobutyric acid.

7. A nutritional supplement for ingestion by humans for restoring growth hormone levels consisting of unmodified rapidly absorbed free-form amino acids, in an amino acid stack which does not include non-amino acid nutritional supplementation, and which, when digested by a user, stimulates release of human growth hormone in the user, said amino acids consisting of the amino acids leucine, isoleucine, valine, lysine, glutamine, ornithine, glycine, arginine and gamma-aminobutyric acid.

8. The nutritional supplement of claim 7, wherein said amino acids in the supplement are to be ingested by humans in a daily dosage, said amino acids in the daily dosage consist of:
(a) Lysine in the mass range 500 mg and 1500 mg;
(b) Arginine in the mass range 500 mg and 1500 mg;
(c) Ornithine in the mass range 500 mg and 1500 mg;
(d) Glutamine in the mass range 200 mg and 1000 mg;
(e) Glycine in the mass range 400 mg and 1000 mg;
(f) Leucine in the mass range 400 mg and 1000 mg;
(g) Iso-leucine in the mass range 400 mg and 1000 mg;
(h) Valine in the mass range 400 mg and 1000 mg; and
(i) Gamma-aminobutyric acid in the mass range 50 mg and 300 mg.

9. The nutritional supplement of claim 8, wherein the amino acid Gamma-aminobutyric acid is in the mass range 100 mg and 200 mg.

10. The nutritional supplement of claim 8, wherein said amino acids in the daily dosage consist of:
(a) 1302 mg of L-Lysine HCl;
(b) 1302 mg of L-Arginine HCl;
(c) 798 mg of L-Ornithine HCl;
(d) 500 mg of Glycine;
(e) 250 mg of L-Glutamine;
(f) 400 mg of L-Leucine;
(g) 400 mg of L-Isoleucine;
(h) 400 mg of L-Valine; and
(i) 100 mg of Gamma-aminobutyric add.

11.
